(19) United States
(12) Patent Application Publication
(10) Pub. No.: US 2014/0220112 A1
(43) Pub. Date: Aug. 7, 2014

(54) TRANSFORMATION OF DRUG CYCLODEXTRIN COMPLEX COMPOSITIONS INTO COMPOSITIONS OF MIXTURES OF LIPID VESICLE ENCAPSULATED DRUG AND CYCLODEXTRIN DRUG COMPLEXES

(71) Applicant: Zoneone Pharma, Inc., San Francisco, CA (US)

(72) Inventors: Francis C. SZOKA, JR., San Francisco, CA (US); Charles O. Noble, San Francisco, CA (US); Mark E. Hayes, San Francisco, CA (US)

(21) Appl. No.: 14/171,728

(22) Filed: Feb. 3, 2014

Related U.S. Application Data
(60) Provisional application No. 61/759,923, filed on Feb. 1, 2013.
(60) Provisional application No. 61/760,410, filed on Feb. 4, 2013.

(51) Int. Cl. A61K 47/48; A61K 31/343; A61K 31/194; A61K 31/439; A61K 31/4745; A61K 38/07; A61K 31/496

(52) U.S. Cl. CPC A61K 47/48815; A61K 38/07; A61K 31/343; A61K 31/496; A61K 31/439; A61K 31/4745; A61K 31/194
USPC 424/450; 514/21.9; 514/469; 514/254.04; 514/253.07; 514/305; 514/283

(57) ABSTRACT

Sparingly water-soluble agents can be formulated as cyclodextrin complexes, however, these water-soluble drug-cyclodextrin complexes dissociate when the complex is administered into patients. The dilution of the complex in the patient leads to the drug being released from the complex, so the drug is not effectively targeted. In contrast, drugs encapsulated in the aqueous core of a lipid vesicles are not released when the liposome is diluted in blood. This invention describes compositions and methods whereby cyclodextrin or polyanionic beta-cyclodextrin drug-complexes are mixed with a preformed liposome containing the amine salts of an acidic compound. This results in the drug cyclodextrin complex being transferred into the liposome where it is stably retained. The liposome-encapsulated drug can then be injected into a patient.

### CROSS-REFERENCE TO RELATED APPLICATIONS

This application claims priority to U.S. Provisional Patent Application No. 61/759,923 filed Feb. 1, 2013 and U.S. Provisional Patent Application No. 61/760,410, filed Feb. 4, 2013, the disclosure of each of which is incorporated herein by reference in its entirety for all purposes.

### BACKGROUND OF THE INVENTION

This invention relates to the fields of pharmaceutical formulation, methods for making them and the uses of the resulting compositions in drug therapy. The pharmaceutical formulations include a mixture of a cyclodextrin complex of a sparingly water-soluble agent on the outside of a lipid vesicle and the uncomplexed sparingly water-soluble agent encapsulated in the interior aqueous compartment of a lipid vesicle. The lipid vesicle and the complexed agent are formulated in a pharmaceutically acceptable diluent.

### DESCRIPTION OF THE RELATED ART

The pharmaceutical industry, in its quest for improved drugs, has generated a large number of potent compounds that are sparingly soluble in water, the ubiquitous solvent that makes life possible. The low water solubility of these new drugs has made it difficult to deliver them in animals including humans. This has created the need for drug delivery systems that can solubilize sparingly water-soluble drugs to enable to their delivery in the body.

The two leading candidate delivery systems for solubilizing sparingly soluble drugs are the cyclodextrins and liposomes. Cyclodextrins (CD) are cyclic oligosaccharides containing six (α-CD), seven (β-CD), eight (γ-CD), or more (a-1,4-)-linked d-glucopyranose units (reviewed by Loftsson T, Brewster M E., Pharmaceutical Applications of Cyclodextrins: Basic Science and Product Development. J. Pharm. Pharmacol. 62(11):1607-21, 2010. The CDs can be visualized as a doughnut with a hydrophilic outside and a hydrophobic interior cavity FIG. 1. A CD will solubilize a drug by forming a complex in which the drug is complexed in the interior cavity. The complex has a relatively low affinity so when the complex is diluted the drug dissociates from the CD. This reduces the solubility of the drug and interferes with the ability of the CD to deliver a drug to a target site in the body after injection.

Lipid vesicles also know as liposomes are vesicle structures usually composed of a bilayer membrane of amphipathic molecules such as, phospholipids, entrapping an aqueous core. The diameters and morphology of various types of liposomes are illustrated in FIG. 2. Drugs can either be encapsulated in the aqueous core or interdigitated in the bilayer membrane. Drugs interdigitated in the membrane transfer out of the liposome when it is diluted into the body, hence in this regard, they have similar drug delivery properties as a CD. Importantly, drugs that are encapsulated in the aqueous core or held in complexes in the aqueous core are retained substantially longer than drugs in the bilayer. The use of liposomes with drugs encapsulated in the aqueous core for drug delivery is well established (Drummond review).

A variety of loading methods for encapsulating functional compounds, particularly drugs, in liposomes is available. Hydrophilic compounds for example can be encapsulated in liposomes by hydrating a mixture of the functional compounds and vesicle-forming lipids. This technique is called passive loading. The functional compound is encapsulated in the liposome as the nanoparticle is formed. The available lipid vesicle (liposome) production procedures are satisfactory for most applications where water-soluble drugs are encapsulated (G. Gregoriadis, Liposome Technology: Liposome Preparation and Related Techniques, 3rd Edition (2006)). However, the manufacture of lipid vesicles that encapsulate drugs sparingly water-soluble (e.g., with a water solubility less than 2 mg/mL) in the aqueous inner compartment of the liposome is exceedingly difficult. This has led to a preference on the part of the pharmaceutical industry for cyclodextrin complexation protocols over liposomes to solubilize sparingly water-soluble drugs for use in disease treatments in patients.

Lipophilic and to a lesser extent amphiphilic functional compounds are loaded somewhat more efficiently than hydrophilic functional compounds because they partition in both the lipid bilayer and the intraliposomal (internal) aqueous medium. However, using passive loading, the final functional-compound-to-lipid ratio as well as the encapsulation efficiency are generally low. The concentration of drug in the liposome equals that of the surrounding fluid and drug not entrapped in the internal aqueous medium is washed away after encapsulation.

US 2009/0196918 A1 discloses liposomal formulations with inclusion complexes of hydroxypropyl-cyclodextrin or sulfobutylether-cyclodextrin and hydrophobic lactone drugs. The cyclodextrin-drug inclusion complex is entrapped into the liposomes via passive loading. Similarly, US 2007/0014845 discloses a liposomal delivery vehicle, including a lipid derivatized with a hydrophilic polymer, for hydrophobic drugs with an aqueous solubility of less than about 50 μg/mL. The encapsulation efficiency of the passive loading techniques is unsatisfactory.

Certain hydrophilic or amphiphilic compounds can be loaded into preformed liposomes using transmembrane pH— or ion-gradients (D. Zucker et al., Journal of Controlled Release (2009) 139:73-80). This technique is called active or remote loading. Compounds amenable to active loading should be able to change from an uncharged form, which can diffuse across the liposomal membrane, to a charged form that is not capable thereof. Typically, the functional compound is loaded by adding it to a suspension of liposomes prepared to have a lower outside/higher inside pH— or ion-gradient. Via active loading, a high functional-compound-to-lipid mass ratio and a high loading efficiency (up to 100%) can be achieved. Examples are active loading of anticancer drugs doxorubicin, daunorubicin, and vincristine (P. R. Cullis et al., Biochimica et Biophysica Acta, (1997) 1331:187-211, and references therein).

Liposomes actively loaded with an active agent from a cyclodextrin complex of the agent have been reported. For example, Gaillard et al. (WO2012/118376) have developed a method for active loading of a water-insoluble drug by first solubilizing the drug in aqueous solution using a solubility enhancing agent. In this method, the solubility enhancing agent remains outside of the liposome during and after loading: very little or no solubilizer is present in the liposomal formulation. Gaillard teaches that liposomal formulations without solubility enhancing agent in them are desirable since these solubility enhancing agents and solubility enhancing conditions can be toxic or irritating towards humans.

To date, a pharmaceutical formulation has not been developed utilizing active loading of the aqueous core of a liposome with a sparingly water-soluble agent from a cyclodextrin complex in a manner in which uncomplexed agent is encapsulated in the interal aqueous medium of the liposome, and a fraction of the cyclodextrin complex remains essentially outside of the aqueous core of the liposome. Thus, in an exemplary embodiment, the presenting invention provides a pharmaceutical formulation mechanisms solubilizing the sparingly water-soluble agent, i.e., cyclodextrin complex on the exterior of the liposome and uncomplexed agent encapsulated in the interior aqueous medium of the liposome. The new formulations represent a significant advance in controlling the rate and location of delivery of sparingly water-soluble agents. The formulations of the invention are readily prepared in the vial of currently approved cyclodextrin complexed drugs by the simple addition of the liposome suspension to the vial.

### SUMMARY OF THE INVENTION

In utilizing liposomes for delivery of functional compounds, it is generally desirable to load the liposomes to high concentration, resulting in a high functional-compound-lipid mass ratio, since this reduces the amount of liposomes to be administered per treatment to attain the required therapeutic effect, all the more since several lipids used in liposomes have a dose-limiting toxicity by themselves. The loading percentage is also of importance for cost efficiency, since poor loading results in a great loss of the active compound.

In an exemplary embodiment, the invention provides a liposome comprising a liposomal lipid membrane encapsulating an internal aqueous medium. The internal aqueous medium comprises an aqueous solution of a complex between a solubility enhancing agent and a first fraction of a sparingly water-soluble agent.

In various embodiments, the invention provides a liposome with two or more fractions of agent within the liposome. An exemplary first fraction includes the agent complexed with a cyclodextrin solubility enhancing agent in the external aqueous medium of the liposome. An exemplary second fraction includes uncomplexed the sparingly water-soluble agent stably incorporated into the interior aqueous compartment of the liposome. In an exemplary embodiment, liposomes with two or more fractions of agent provide distribution profiles that are bi- or higher-modal. For example, in one embodiment, complexed agent is released from the external aqueous medium at a faster rate than uncomplexed agent within the lipid aqueous interior providing a therapeutic mixture with a bimodal release kinetics.

In a further exemplary embodiment, the invention provides pharmaceutical formulations comprising a liposome of the invention. The formulations include the liposome and a pharmaceutically acceptable diluent or excipient. In various embodiments, the pharmaceutical formulation is in a unit dosage format, providing a unit dosage of the therapeutic agent encapsulated in the liposome.

In another exemplary embodiment, the invention provides methods of making the liposomes of the invention. In various embodiments, there is provided a method comprising: a) incubating an aqueous mixture comprising: (i) liposomes having a liposomal lipid membrane encapsulating an internal aqueous medium; (ii) a complex between a solubility enhancing agent and a first fraction of the sparingly water-soluble substance; and (iii) an external aqueous medium. The mixture used to load the liposome with the agent (or complexed agent) is prepared such that a proton- and/or ion-gradient exists across the liposomal membrane between the internal aqueous membrane and the external aqueous medium. The incubating can be for any useful period but is preferably for a period of time sufficient to cause at least part of the complex to be drawn out of the external aqueous medium and to accumulate in the internal aqueous medium under the influence of the proton and/or ion gradient.

Other embodiments, objects and advantages are set forth in the Detailed Description that follows.

### BRIEF DESCRIPTION OF THE DRAWINGS

FIG. 1 illustrates CDs that can be visualized as a doughnut with a hydrophilic outside and a hydrophobic interior cavity.

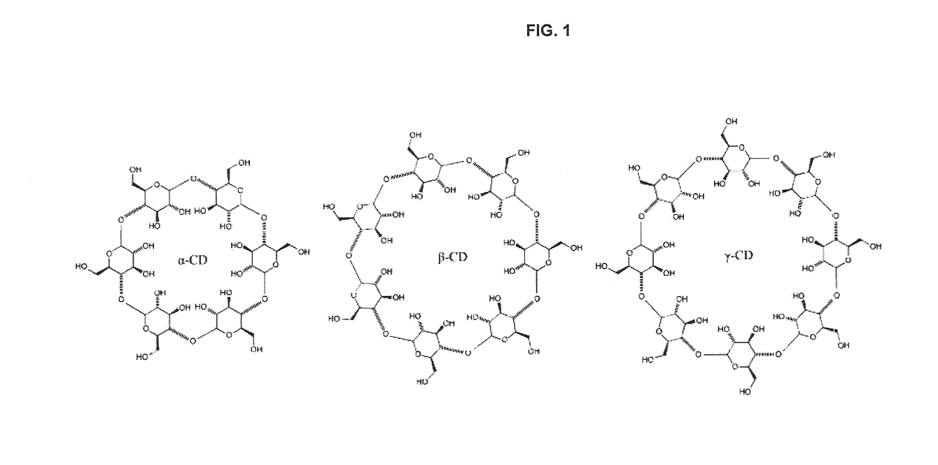

FIG. 2 illustrates the diameters and morphology of various types of liposomes.

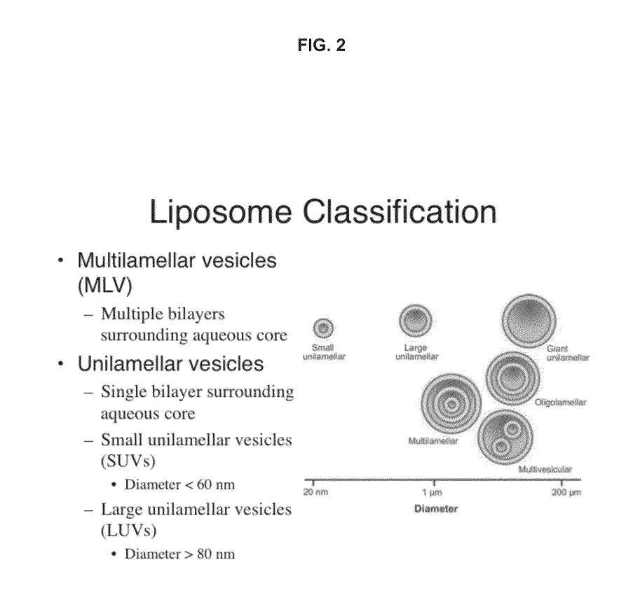

### DESCRIPTION OF THE PREFERRED EMBODIMENTS

### Introduction

In utilizing liposomes for delivery of functional compounds, it is generally desirable to load the liposomes to high concentration, resulting in a high agent-lipid mass ratio, since this reduces the amount of liposomes to be administered per treatment to attain the required therapeutic effect of the agent, all the more since several lipids used in liposomes have a dose-limiting toxicity by themselves. The loading percentage is also of importance for cost efficiency, since poor loading results in an increase loss of agent during the loading of the agent into the liposome.

The final agent-to-lipid ratio for agents sparingly soluble in water can be increased using solubility enhancing agents to increase the concentration of the sparingly water-soluble agent in the extraliposomal aqueous medium. Many methods, based on the use of co-solvents, surfactants, and complexing agents, for solubilizing lipophilic compounds in water have been developed. Several commonly used solubility enhancing agents in aqueous agent formulations are cyclodextrins, propylene glycol, polyethylene glycols, ethanol, sorbitol, non-ionic surfactants, and polyethoxylated castor oil.

The present invention provides liposomes encapsulating solubilized agents, methods of making such liposomes, formulations containing such liposomes and methods of making the liposomes and formulations of the invention.

In an exemplary embodiment, the invention provides a pharmaceutical formulation comprising a liposome having a membrane encapsulating an aqueous compartment. Encapsulated within the aqueous compartment is the uncomplexed sparingly water-soluble therapeutic agent and a salt of a remote loading agent. In various embodiments, about 20%, about 30%, about 50%, about 70% or about 90% of the sparingly water-soluble agent is encapsulated within the aqueous compartment of the liposome. In various embodiments, the agent-cyclodextrin is dissolved in the external aqueous compartment to a concentration of from about 0.05 mM to about 2 mM.

In some embodiments, essentially the entire amount of the agent-component of cyclodextrin complex is concentrated within the aqueous compartment of the liposome and little to essentially none is external to the liposome or interdigitated within the lipid bilayer. In an exemplary embodiment, at least about 95%, at least about 90%, at least about 85%, at least about 80%, at least about 50, or at least about 30% of the agent from the cyclodextrin complex is encapsulated within the aqueous compartment of the liposome.

In an exemplary embodiment, the sparingly water-soluble therapeutic agent is not covalently attached to the cyclodextrin or to a component of the liposome.

### Liposomes

The term liposome is used herein in accordance with its usual meaning, referring to microscopic lipid vesicles composed of a bilayer of phospholipids or any similar amphipathic lipids encapsulating an internal aqueous medium. The liposomes of the present invention can be unilamellar vesicles such as small unilamellar vesicles (SUVs) and large unilamellar vesicles (LUVs), and multilamellar vesicles (MLV), typically varying in size from 50 nm to 200 nm. No particular limitation is imposed on the liposomal membrane structure in the present invention. The term liposomal membrane refers to the bilayer of phospholipids separating the internal aqueous medium from the external aqueous medium.

Exemplary liposomal membranes useful in the current invention may be formed from a variety of vesicle-forming lipids, typically including dialiphatic chain lipids, such as phospholipids, diglycerides, dialiphatic glycolipids, single lipids such as sphingomyelin and glycosphingolipid, cholesterol and derivates thereof, and combinations thereof. As defined herein, phospholipids are amphiphilic agents having hydrophobic groups formed of long-chain alkyl chains, and a hydrophilic group containing a phosphate moiety. The group of phospholipids includes phosphatidic acid, phosphatidyl glycerols, phosphatidylcholines, phosphatidylethanolamines, phosphatidylinositols, phosphatidylserines, and mixtures thereof. Preferably, the phospholipids are chosen from 1,2-dipalmitoyl-sn-glycero-3-phosphocholine (DPPC), dimyristoyl-phosphatidylcholine (DMPC), hydrogenated soy phosphatidylcholine (HSPC), soy phosphatidylcholine (SPC), distearoyl phosphatidylcholine (DSPC), egg yolk phosphatidylcholine (EYPC) or hydrogenated egg yolk phosphatidylcholine (HEPC), distearoylphosphatidylglycerol (DSPG), sterol modified lipids, cationic lipids and zwitterlipids

Liposomal membranes according to the present invention may further comprises ionophores like nigericin and A23187.

In the method according to the present invention, an exemplary liposomal phase transition temperature is between −20° C. and 100° C., e.g., between −20° C. and 65° C. The phase transition temperature is the temperature required to induce a change in the physical state of the lipids constituting the liposome, from the ordered gel phase, where the hydrocarbon chains are fully extended and closely packed, to the disordered liquid crystalline phase, where the hydrocarbon chains are randomly oriented and fluid. Above the phase transition temperature of the liposome, the permeability of the liposomal membrane increases. Choosing a high transition temperature, where the liposome would always be in the gel state, could provide a non-leaking liposomal composition, i.e. the concentration of the sparingly water-soluble agent in the internal aqueous medium is maintained during exposure to the environment. Alternatively, a liposome with a transition temperature between the starting and ending temperature of the environment it is exposed to provides a means to release the sparingly water-soluble agent when the liposome passes through its transition temperature. Thus, the process temperature for the active-loading technique typically is above the liposomal phase transition temperature to facilitate the active-loading process. As is generally known in the art, phase transition temperatures of liposomes can, among other parameters, be influenced by the choice of phospholipids and by the addition of steroids like cholesterol, lanosterol, cholestanol, stigmasterol, ergosterol, and the like. Hence, in an embodiment of the invention, a method according to any of the foregoing is provided in which the liposomes comprise one or more components selected from different phospholipids and cholesterol in several molar ratios in order to modify the transition, the required process temperature and the liposome stability in plasma. Less cholesterol in the mixture will result in less stable liposomes in plasma. An exemplary phospholipid composition of use in the invention comprises between about 10 and about 50 mol % of steroids, preferably cholesterol.

In accordance with the invention, liposomes can be prepared by any of the techniques now known or subsequently developed for preparing liposomes. For example, the liposomes can be formed by the conventional technique for preparing multilamellar lipid vesicles (MLVs), that is, by depositing one or more selected lipids on the inside walls of a suitable vessel by dissolving the lipids in chloroform and then evaporating the chloroform, and by then adding the aqueous solution which is to be encapsulated to the vessel, allowing the aqueous solution to hydrate the lipid, and swirling or vortexing the resulting lipid suspension. This process engenders a mixture including the desired liposomes. Alternatively, techniques used for producing large unilamellar lipid vesicles (LUVs), such as reverse-phase evaporation, infusion procedures, and detergent dilution, can be used to produce the liposomes. A review of these and other methods for producing lipid vesicles can be found in the text Liposome Technology, Volume I, Gregory Gregoriadis Ed., CRC Press, Boca Raton, Fla., (1984), which is incorporated herein by reference. For example, the lipid-containing particles can be in the form of steroidal lipid vesicles, stable plurilamellar lipid vesicles (SPLVs), monophasic vesicles (MPVs), or lipid matrix carriers (LMCs). In the case of MLVs, if desired, the liposomes can be subjected to multiple (five or more) freeze-thaw cycles to enhance their trapped volumes and trapping efficiencies and to provide a more uniform interlamellar distribution of solute.

Following liposome preparation, the liposomes are optionally sized to achieve a desired size range and relatively narrow distribution of liposome sizes. A size range of about 20-200 nanometers allows the liposome suspension to be sterilized by filtration through a conventional filter, typically a 0.22 or 0.4 micron filter. The filter sterilization method can be carried out on a high through-put basis if the liposomes have been sized down to about 20-200 nanometers. Several techniques are available for sizing liposomes to a desired size. Sonicating a liposome suspension either by bath or probe sonication produces a progressive size reduction down to small unilamellar vesicles less than about 50 nanometers in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 50 and 500 nanometers, are observed. In both methods, the particle size distribution can be monitored by conventional laser-beam particle size determination. Extrusion of liposome through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve a gradual reduction in liposome size. Alternatively limit size liposomes can be prepared using microfluidic techniques wherein the lipid in an organic solvent such as ethanol or ethanol-aprotic solvent mixtures is rapidly mixed with the aqueous medium, so that the organic solvent/water ratio is less than 30%, in a microchannel with dimensions less than 300 microns and preferable less than 150 microns in wide and 50 microns in height. The organic solvent is then removed from the liposomes by dialysis. Other useful sizing methods such as sonication, solvent vaporization or reverse phase evaporation are known to those of skill in the art.

Exemplary liposomes for use in various embodiments of the invention have a size of from about 30 nanometers to about 40 microns. In an exemplary embodiment, the liposomes are from about 40 nm to about 150 nm in diameter.

The internal aqueous medium, as referred to herein, typically is the original medium in which the liposomes were prepared and which initially becomes encapsulated upon formation of the liposome. In accordance with the present invention, freshly prepared liposomes encapsulating the original aqueous medium can be used directly for active loading. Embodiments are also envisaged however wherein the liposomes, after preparation, are dehydrated, e.g. for storage. In such embodiments the present process may involve addition of the dehydrated liposomes directly to the external aqueous medium used to create the transmembrane gradients. However it is also possible to hydrate the liposomes in another external medium first, as will be understood by those skilled in the art. Liposomes are optionally dehydrated under reduced pressure using standard freeze-drying equipment or equivalent apparatus. In various embodiments, the liposomes and their surrounding medium are frozen in liquid nitrogen before being dehydrated and placed under reduced pressure. To ensure that the liposomes will survive the dehydration process without losing a substantial portion of their internal contents, one or more protective sugars are typically employed to interact with the lipid vesicle membranes and keep them intact as the water in the system is removed. A variety of sugars can be used, including such sugars as trehalose, maltose, sucrose, glucose, lactose, and dextran. In general, disaccharide sugars have been found to work better than monosaccharide sugars, with the disaccharide sugars trehalose and sucrose being most effective. Other more complicated sugars can also be used. For example, aminoglycosides, including streptomycin and dihydrostreptomycin, have been found to protect liposomes during dehydration. Typically, one or more sugars are included as part of either the internal or external media of the lipid vesicles. Most preferably, the sugars are included in both the internal and external media so that they can interact with both the inside and outside surfaces of the liposomes' membranes. Inclusion in the internal medium is accomplished by adding the sugar or sugars to the buffer which becomes encapsulated in the lipid vesicles during the liposome formation process. In these embodiments the external medium used during the active loading process should also preferably include one or more of the protective sugars

As is generally known to those skilled in the art, polyethylene glycol (PEG)-lipid conjugates have been used extensively to improve circulation times for liposome-encapsulated functional compounds, to avoid or reduce premature leakage of the functional compound from the liposomal composition and to avoid detection of liposomes by the body's immune system. Attachment of PEG-derived lipids onto liposomes is called PEGylation. Hence, in an exemplary embodiment of the invention, the liposomes are PEGylated liposomes. PEGylation can be accomplished by incubating a reactive derivative of PEG with the target liposomes. Suitable PEG-derived lipids according to the invention, include conjugates of DSPE-PEG, functionalized with one of carboxylic acids, glutathione (GSH), maleimides (MAL), 3-(2-pyridyldithio) propionic acid (PDP), cyanur, azides, amines, biotin or folate, in which the molecular weight of PEG is between 2000 and 5000 g/mol. Other suitable PEG-derived lipids are mPEGs conjugated with ceramide, having either C₈- or C₁₆-tails, in which the molecular weight of mPEG is between 750 and 5000 daltons. Still other appropriate ligands are mPEGs or functionalized PEGs conjugated with glycerophospholipds like 1,2-dimyristoyl-sn-glycero-3-phosphoethanolamine (DMPE), 1,2-dipalmitoyl-sn-glycero-3-phosphoethanolamine (DPPE), 1,2-dioleoyl-sn-glycero-3-phosphoethanolamine (DOPE) and 1,2-distearoyl-sn-glycero-3-phosphoethanolamine (DSPE), and the like. PEGylation of liposomes is a technique generally known by those skilled in the art.

In various embodiments, the liposomes are PEGylated with DSPE-PEG-GSH conjugates (up to 5 mol %) and/or DSPE-mPEG conjugates (wherein the molecular weight of PEG is typically within the range of 750-5000 daltons, e.g. 2000 daltons). The phospholipid composition of an exemplary PEGylated lipsome of the invention may comprise up to 5-20 mol % of PEG-lipid conjugates.

Furthermore, in certain embodiments, one or more moieties that specifically target the liposome to a particular cell type, tissue or the like are incorporated into the membrane. Targeting of liposomes using a variety of targeting moieties (e.g., ligands, receptors and monoclonal antibodies) has been previously described. Suitable examples of such targeting moieties include: hyaluronic acid, lipoprotein lipase (LPL), [α]2-macroglobulin ([α]2M), receptor associated protein (RAP), lactoferrin, desmoteplase, tissue- and urokinase-type plasminogen activator (tPA/uPA), plasminogen activator inhibitor (PAI-I), tPA/uPA:PAI-1 complexes, melanotransferrin (or P97), thrombospondin 1 and 2, hepatic lipase, factor Vila/tissue-factor pathway inhibitor (TFPI), factor VIIIa, factor IXa, A[β]1-40, amyloid-[β] precursor protein (APP), CI inhibitor, complement C3, apolipoproteinE (apoE), pseudomonas exotoxin A, CRM66, HIV-I Tat protein, rhinovirus, matrix metalloproteinase 9 (MMP-9), MMP-13 (collagenase-3), sphingolipid activator protein (SAP), pregnancy zone protein, antithrombin III, heparin cofactor II, [α]1-antitrypsin, heat shock protein 96 (HSP-96), platelet-derived growth factor (PDGF), apolipoproteinJ (apoJ, or clusterin), A[β] bound to apoJ and apoE, aprotinin, angiopep-2 (TFFYGGSRGKRNNFKTEEY), very-low-density lipoprotein (VLDL), transferrin, insulin, leptin, an insulin-like growth factor, epidermal growth factors, lectins, peptidomimetic and/or humanized monoclonal antibodies or peptides specific for said receptors (e.g., sequences HAIYPRH and THRPPMWSPVWP that bind to the human transferrin receptor, or anti-human transferrin receptor (TfR) monoclonal antibody A24), hemoglobin, non-toxic portion of a diphtheria toxin polypeptide chain, all or a portion of the diphtheria toxin B chain, all or a portion of a non-toxic mutant of diphtheria toxin CRM197, apolipoprotein B, apolipoprotein E (e.g., after binding to polysorb-80 coating), vitamin D-binding protein, vitamin A/retinol-binding protein, vitamin B12/cobalamin plasma carrier protein, glutathione and transcobalamin-B 12.

Targeting mechanisms generally require that the targeting agents be positioned on the surface of the liposome in such a manner that the target moieties are available for interaction with the target, for example, a cell surface receptor. In an exemplary embodiment, the liposome is manufactured to include a connector portion incorporated into the membrane at the time of forming the membrane. An exemplary connector portion has a lipophilic portion which is firmly embedded and anchored in the membrane. An exemplary connector portion also includes a hydrophilic portion which is chemically available on the aqueous surface of the liposome. The hydrophilic portion is selected so that it will be chemically suitable to form a stable chemical bond with the targeting agent, which is added later. Techniques for incorporating a targeting moiety in the liposomal membrane are generally known in the art.

### Sparingly Water-Soluble Agent

As indicated above, the present invention provides liposomes encapsulating a a sparingly water-soluble agent. In the context of the present invention the term ‘sparingly water-soluble’ means being insoluble or having a very limited solubility in water, more in particular having an aqueous solubility of less than 2 mg/mL, e.g., less than 1.9 mg/mL, e.g., having an aqueous solubility of less than 1 mg/mL. As used herein, water solubilities refer to solubilities measured at ambient temperature, which is typically about 20° C. In an exemplary embodiment, the water solubility of the agent is measured at about pH=7.

According to an exemplary embodiment of the invention, the sparingly water-soluble agent is a therapeutic agent selected from the group of a therapeutic is selected from a group consisting of an anthracycline compound, a camptothecin compound, a vinca alkaloid, an ellipticine compound, a taxane compound, a wortmannin compound, a geldanamycin compound, a pyrazolopyrimidine compound, a peptide-based compound such as carfilzomib, a steroid compound, a derivative of any of the foregoing, a pro-drug of any of the foregoing, and an analog of any of the foregoing.

Exemplary small molecule compounds having a water solubility less than about 2 mg/mL include, but are not limited to, carfilzomib, voriconazole, amiodarone, ziprasidone, aripiprazole, imatinib, lapatinib, cyclopamine, oprozomib, CUR-61414, PF-05212384, PF-4691502, toceranib, PF-477736, PF-337210, sunitinib, SU14813, axitinib, AG014699, veliparib, MK-4827, ABT-263, SU11274, PHA665752, Crizotinib, XL880, PF-04217903, XR5000, AG14361, veliparib, bosutunib, PD-0332991, PF-01367338, AG14361, NVP-ADW742, NVP-AUY922, NVP-LAQ824, NVP-TAE684, NVP-LBH589, erubulin, doxorubicin, daunorubicin, mitomycin C, epirubicin, pirarubicin, rubidomycin, carcinomycin, N-acetyladriamycin, rubidazone, 5-imido daunomycin, N-acetyl daunomycin, daunory line, mitoxanthrone, camptothecin, 9-aminocamptothecin, 7-ethylcamptothecin, 7-Ethyl-10-hydroxy-camptothecin, 10-hydroxycamptothecin, 9-nitrocamptothecin,1O,11-methylenedioxycamptothecin, 9-amino-1O,11-methylenedioxycamptothecin, 9-chloro-10, 11-methylenedioxycamptothecin, irinotecan, lurtotecan, silatecan, (7-(4-methylpiperazinomethylene)-10,11-ethylenedioxy-20(S)-camptothecin, 7-(4-methylpiperazinomethylene)-10, II-methylenedioxy-20(S)-camptothecin, 7-(2-N-isopropylamino)ethyl)-(20S)-camptothecin, CKD-602, vincristine, vinblastine, vinorelbine, vinflunine, vinpocetine, vindesine, ellipticine, 6-3-aminopropyl-ellipticine, 2-diethylaminoethyl-ellipticinium, datelliptium, retelliptine, paclitaxel, docetaxel, diclofenac, bupivacaine, 17-Dimethylaminoethylamino-17-demethoxygeldanamycin, cetirizine, fexofenadine, primidone and other catecholamines, epinephrine, (S)-2-(2,4-dihydroxyphenyl)-4,5-dihydro-4-methyl-4-thiazolecarboxylic acid (deferitrin), (S)-4,5-dihydro-2-(3-hydroxy-2-pyridinyl)-4-methyl-4-thiazolecarboxylic acid (desferrithiocin), (S)-4,5-dihydro-2-[2-hydroxy-4-(3,6,9,12-tetraoxamidecyloxy)phenyl]-4-methyl-4-thiazolecarboxylic acid, (S)-4,5-dihydro-2-[2-hydroxy-4-(3,6-dioxaheptyloxy)phenyl]-4-methyl-4-thiazolecarboxylic acid, ethyl (S)-4,5-dihydro-2-[2-hydroxy-4-(3,6-dioxaheptyloxy)phenyl]-4-methyl-4-thiazolecarboxylate, (S)-4,5-dihydro-2-[2-hydroxy-3-(3,6,9-trioxadecyloxy)]-4-methyl-4-thiazolecarboxylic acid, desazadesferrithiocin salts, prodrugs and derivatives of these medicinal compounds and mixtures thereof.

An exemplary therapeutic agent is selected from: an antihistamine ethylenediamine derivative, bromphenifamine, diphenhydramine, an anti-protozoal drug, quinolone, iodoquinol, an amidine compound, pentamidine, an antihelmintic compound, pyrantel, an anti-schistosomal drug, oxaminiquine, an antifungal triazole derivative, fliconazole, itraconazole, ketoconazole, miconazole, an antimicrobial cephalosporin, chelating agents, deferoxamine, deferasirox, deferiprone, FBS0701, cefazolin, cefonicid, cefotaxime, ceftazimide, cefuoxime, an antimicrobial beta-lactam derivative, aztreopam, cefmetazole, cefoxitin, an antimicrobial of erythromycin group, erythromycin, azithromycin, clarithromycin, oleandomycin, a penicillin compound, benzylpenicillin, phenoxymethylpenicillin, cloxacillin, methicillin, nafcillin, oxacillin, carbenicillin, a tetracycline compound, novobiocin, spectinomycin, vancomycin; an antimycobacterial drug, aminosalicycic acid, capreomycin, ethambutol, isoniazid, pyrazinamide, rifabutin, rifampin, clofazimine, an antiviral adamantane compound, amantadine, rimantadine, a quinidine compound, quinine, quinacrine, chloroquine, hydroxychloroquine, primaquine, amodiaquine, mefloquine, an antimicrobial, qionolone, ciprofloxacin, enoxacin, lomefloxacin, nalidixic acid, norfloxacin, ofloxacin, a sulfonamide; a urinary tract antimicrobial, nitrofurantoin, trimetoprim; anitroimidazoles derivative, metronidazole, a cholinergic quaternary ammonium compound, ambethinium, neostigmine, physostigmine, an anti-Alzheimer aminoacridine, tacrine, an anti-parkinsonal drug, benztropine, biperiden, procyclidine, trihexylhenidyl, an anti-muscarinic agent, atropine, hyoscyamine, scopolamine, propantheline, an adrenergic compound, dopamine, serotonin, a hedgehog inhibitor, albuterol, dobutamine, ephedrine, epinephrine, norepinephrine, isoproterenol, metaproperenol, salmetrol, terbutaline, a serotonin reuptake inhibitor, an ergotamine derivative, a myorelaxant, a curare series, a central action myorelaxant, baclophen, cyclobenzepine, dentrolene, nicotine, a nicotine receptor antagonist, a beta-adrenoblocker, acebutil, amiodarone, abenzodiazepine compound, ditiazem, an antiarrhythmic drug, diisopyramide, encaidine, a local anesthetic compound, procaine, procainamide, lidocaine, flecaimide, quinidine, an ACE inhibitor, captopril, enelaprilat, Hsp90 inhibitor, fosinoprol, quinapril, ramipril; an opiate derivative, codeine, meperidine, methadone, morphine, an antilipidemic, fluvastatin, gemfibrosil, an HMG-coA inhibitor, pravastatin, a hypotensive drug, clonidine, guanabenz, prazocin, guanethidine, granadril, hydralazine, a non-coronary vasodilator, dipyridamole, an acetylcholine esterase inhibitor, pilocarpine, an alkaloid, physostigmine, neostigmine, a derivative of any of the foregoing, a pro-drug of any of the foregoing, and analog of any of the foregoing.

This list of agents, however, is not intended to limit the scope of the invention. In fact, the compound encapsulated within the liposome can be any sparingly water-soluble amphipathic weak base or amphipathic weak acid. As noted above, embodiments wherein the sparingly water-soluble agent is not a pharmaceutical or medicinal agent are also encompassed by the present invention.

Typically, within the context of the present invention, sparingly water-soluble amphipathic weak bases have an octanol-water distribution coefficient (log D) at pH 7 between −2.5 and 2 and pKa <11, while sparingly water-soluble amphipathic weak acids have a log D at pH 7 between −2.5 and 2 and pKa >3.

Typically, the terms weak base and weak acid, as used in the foregoing, respectively refer to compounds that are only partially protonated or deprotonated in water. Examples of protonable agents include compounds having an amino group, which can be protonated in acidic media, and compounds which are zwitterionic in neutral media and which can also be protonated in acidic environments. Examples of deprotonable agents include compounds having a carboxy group, which can be deprotonated in alkaline media, and compounds which are zwitterionic in neutral media and which can also be deprotonated in alkaline environments.

The term zwitterionic refers to compounds that can simultaneously carry a positive and a negative electrical charge on different atoms. The term amphipathic, as used in the foregoing is typically employed to refer to compounds having both lipophilic and hydrophilic moieties. The foregoing implies that aqueous solutions of compounds being weak amphipathic acids or bases simultaneously comprise charged and uncharged forms of said compounds. Only the uncharged forms may be able to cross the liposomal membrane.

When agents of use in the present invention contain relatively basic or acidic functionalities, salts of such compounds are included in the scope of the invention. Salts can be obtained by contacting the neutral form of such compounds with a sufficient amount of the desired acid or base, either neat or in a suitable inert solvent. Examples of salts for relative acidic compounds of the invention include sodium, potassium, calcium, ammonium, organic amino, or magnesium salts, or a similar salts. When compounds of the present invention contain relatively basic functionalities, acid addition salts can be obtained by contacting the neutral form of such compounds with a sufficient amount of the desired acid, either neat or in a suitable inert solvent. Examples of acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science 1977, 66: 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

An exemplary agent is a small organic molecule with a molecular weight between about 100 Da and 3000 Da.

In the embodiment in which a unit dosage format is formed, the liposome will, in exemplary embodiments include from about 1 mg to about 500 mg of the approved agent, e.g, from about 1 mg to about 200 mg, e.g., from about 5 mg to about 100 mg, e.g., from about 10 mg to about 60 mg.

In an exemplary embodiment, the unit dosage includes the approved agent carfilzomib and it is present in the liposome in an amount of from about 40 mg to about 80 mg, e.g., from about 50 mg to about 70 mg. In an exemplary embodiment, the carfilzomib is present in about 60 mg.

### Active Loading

As indicated above, the pre-formed liposomes are loaded with a complex between a solubility enhancing agent and the sparingly water-soluble agent according to an active or remote loading technique. The process of active loading, involves the use of transmembrane potentials. The principle of active loading, in general, has been described extensively in the art. The terms active-loading and remote-loading are synonymous and will be used interchangeably.

During active loading, the complex of the solubility enhancing agent and the sparingly water-soluble agent is transferred from the external aqueous medium across the liposomal membrane to the internal aqueous medium by a transmembrane proton- or ion-gradient. The term gradient of a particular compound as used herein refers to a discontinuous increase of the concentration of said compound across the liposomal membrane from outside (external aqueous medium) to inside the liposome (internal aqueous medium).

To create the concentration gradient, the liposomes are typically formed in a first liquid, typically aqueous, phase, followed by replacing or diluting said first liquid phase. The diluted or new external medium has a different concentration of the charged species or a totally different charged species, thereby establishing the ion- or proton-gradient.

The replacement of the external medium can be accomplished by various techniques, such as, by passing the lipid vesicle preparation through a gel filtration column, e.g., a Sephadex or Sepharose column, which has been equilibrated with the new medium, or by centrifugation, dialysis, or related techniques.

The efficiency of active-loading into liposomes depends, among other aspects, on the chemical properties of the complex to be loaded and the type and magnitude of the gradient applied. In an embodiment of the invention, a method as defined in any of the foregoing is provided employing a gradient across the liposomal membrane, in which the gradient is chosen from a pH-gradient, a sulfate-, phosphonate-, phosphate-, citrate-, or acetate-salt gradient, an EDTA-ion gradient, an ammonium-salt gradient, an alkylated, e.g methyl-, ethyl-, propyl- and amyl, ammonium-salt gradient, a triethylammonium salt gradient, a Mn²⁺-, Cu²⁺, Na⁺, K⁺-gradient, with or without using ionophores, or a combination thereof. These loading techniques have been extensively described in the art.

Preferably, the internal aqueous medium of pre-formed, i.e. unloaded, liposomes comprises a so-called active-loading buffer which contains water and, dependent on the type of gradient employed during active loading, may further comprise a sulfate-, phosphonate-, phosphate-, citrate-, or acetate-salt, an ammonium-salt, an alkylated, e.g methyl-, ethyl-, propyl- and amyl, ammonium-salt, a Mn²⁺, Cu²⁺ or Na⁺/K⁺-salt, an EDTA-ion salt, and optionally a pH-buffer to maintain a pH-gradient. In an exemplary embodiment, the concentration of salts in the internal aqueous medium of unloaded liposomes is between 1 and 1000 mM.

Exemplary amines of use in the present invention include, without limitation, monoamines, polyamines, trimethylammonium, triethylammonium, tributyl ammonium, diethylmethylammonium, diisopropylethyl ammonium, triisopropylammonium, N-methylmorpholinium, N-ethylmorpholinium, N-hydroxyethylpiperidinium, N-methylpyrrolidinium, N,N-dimethylpiperazinium, isopropylethylammonium, isopropylmethylammonium, diisopropylammonium, tert-butylethylammonium, dicychohexylammonium, protonized forms of morpholine, pyridine, piperidine, pyrrolidine, piperazine, imidazole, tert-butylamine, 2-amino-2-methylpropanol-1,2-amino-2-methyl-propandiol-1,3, and tris-(hydroxyethyl)-aminomethane, diethyl-(2-hydroxyethyl)amine, tris-(hydroxymethyl)-aminomethane tetramethylammonium, tetraethylammonium, N-methylglucamine and tetrabutylammonium, polyethyleneimine, and polyamidoamine dendrimers.

Exemplary carboxylates of use in the invention include, without limitation, citrate, diethylenetriaminepentaaceetate, melletic acetate, 1,2,3,4-butanetetracarboxylate, benzoate, isophalate, phthalate, 3,4-bis(carboxymethyl)cyclopentanecarboxylate, benzenetricarboxylates, the carboxylate generation of polyamidoamine dendrimers, benzenetetracarboxylates, ascorbate, glucuronate, and ulosonate.

Exemplary sulfates include, without limitation, sulfate, 1,5-naphthalenedisulfonate, dextran sulfate, sucrose octasulfate benzene sulfonate, sulfobbutylether beta cyclodextrin, poly(4-styrenesulfonate) trans resveratrol-trisulfate, and sulfobutyletherbetacyclodextrn.

Exemplary phosphates include, but are not limited to: phosphate, hexametaphosphate, phosphate glasses, polyphosphates, triphosphate, trimetaphosphate, bisphosphonates, ethanehydroxy bisphosphonate, and inositol hexaphosphate

Exemplary salts may include one or more of a carboxylate, sulfate or phosphate including but not limited to: 2-carboxybenensulfonate, creatine phosphate, phosphocholine, carnitine phosphate, and the carboxyl generation of polyamidoamines.

The external aqueous medium, used to establish the transmembrane gradient for active loading, comprises water, solubility enhancing agent, the sparingly water-soluble agent(s) to be loaded, and optionally sucrose to adjust the osmolarity and/or a chelator like EDTA to aid ionophore activity, more preferably sucrose and/or EDTA. Saline may also be used to adjust osmolarity. Sucrose can also be used to adjust osmolarity. In a preferred embodiment of the invention a method for actively loading liposomes is provided wherein concentrations of the gradient-forming compound in the internal aqueous medium, and concentrations of the sparingly water-soluble agent(s) and solubility enhancing agent in the external medium are established of such magnitude that net transport of the sparingly water-soluble agent(s) across the liposomal membrane occurs during active loading.

In an exemplary embodiment the gradient is chosen from a pH-, ammonium sulfate- and calcium acetate-gradient. As is generally known by those skilled in the art, transmembrane pH-(lower inside, higher outside pH) or calcium acetate-gradients can be used to actively load amphiphilic weak acids. Amphipathic weak bases can also be actively loaded into liposomes using an ammonium sulfate- or ammonium chloride-gradient.

Depending upon the permeability of the lipid vesicle membranes, the full transmembrane potential corresponding to the concentration gradient will either form spontaneously or a permeability enhancing agent, e.g., a proton ionophore can be added to the medium. If desired, the permeability enhancing agent can be removed from the liposome preparation after loading with the complex is complete using chromatography or other techniques.

Typically the temperature of the medium during active loading is between about 0° C. and about 100° C., e.g., between about 0° C. and about 70° C., e.g., between about 4° C. and 65° C.

The encapsulation or loading efficiency, defined as encapsulated amount (e.g., as measured in moles) of the complex between the solubility enhancing agent and the sparingly water-soluble agent in the internal aqueous phase divided by the initial amount of moles of complex in the external aqueous phase multiplied by 100%, is at least 25%, preferably at least 50%, at least 60%, or at least 70%.

### Solubility Enhancing Agent

As noted hereinbefore, in exemplary embodiments of the invention a complex between an agent and a solubility enhancing agent is added to the external aqueous medium of a liposome preparation to increase the rate and efficiency of uptake of the sparingly water-soluble agent from the external medium into the aqueous compartment of the liposome. The invention provides liposomes having complexes between agents and solubility enhancing agents encapsulated within the aqueous compartment of a liposome.

According to an embodiment of the present invention, a method as defined in the foregoing is provided using a solubility enhancing agent chosen from complexing agents, co-solvents, surfactants and emulsifiers. The solubility enhancing agent typically increases the solubility of the sparingly water-soluble compound in the external aqueous medium at least two-fold, preferably at least three-fold, preferably to values above about 1.9 mM at ambient temperature, e.g., values above about 3.8 mM.

Complexing agents, as this term is utilized herein, are solubility enhancing agents, which are water-soluble compounds that form water-soluble inclusion complexes with the sparingly water-soluble agent, hence increasing the aqueous solubility of the sparingly water-soluble compound. In an embodiment of the present invention, the solubility enhancing agent is a complexing agent chosen from cyclodextrins and polyvinylpyrrolidones (povidones).

Povidones form water-soluble complexes with many functional compounds. Cyclodextrins are also well known in the art for their ability to form stable non-covalent inclusion complexes with a large variety of amphiphilic and lipophilic guest molecules (R. Challa et al., AAPS PharmSciTech, (2005) 6(2) E329-E357). Cyclodextrins have a lipophilic inner cavity and a hydrophilic outer surface providing them with good aqueous solubility. The 3 naturally occurring cyclodextrins, α-, β-, and γ-cyclodextrin differ in their ring size and aqueous solubility. Of these naturally occurring cyclodextrins, the lipophilic inner cavity of β-cyclodextrin is most suitable for complexing a variety of functional compounds. Chemical modification with hydroxy propyl and sulfoalkylether groups increases the aqueous solubility and complexing activity of the naturally occurring cyclodextrins (Loftsson T., Brewster M E., Pharmaceutical Applications of Cyclodextrins: Basic Science and Product Development, J. Pharm. Pharmacol. 2010, 62(11):1607-1621). In an embodiment of the invention the solubilizing agent is chosen from α-, β-, and γ-cyclodextrin and cyclodextrins modified with alkyl-, hydroxyalkyl-, dialkyl-, and preferably sulfoalkyl-ether modified cyclodextrins.

In an exemplary embodiment, the solubility enhancing agent is a complexing agent chosen from β-cyclodextrin, hydroxypropyl-cyclodextrin, and sulfobutylether-β-cyclodextrin.

In the event that the solubility enhancing agent is a complexing agent, it is preferred that the rate of dissociation of the complexing agent and the sparingly water-soluble agent in the external medium is equal to or less than the rate of uptake of the sparingly-water soluble agent from the external medium into the liposome. Without wishing to be bound by any particular theory, it is believed that the former can be established by optimizing the concentrations and/or combinations of complexing agent and sparingly water-soluble agent in the external medium as well as the proton- and/or ion-gradient across the liposomal membrane. Hence, in a preferred embodiment, a method for loading pre-formed liposomes according to any of the foregoing is provided wherein the processing temperature during active loading, the phase transition temperature of the liposomes, the concentrations and/or combinations of complexing agent and sparingly water-soluble agent in the external medium as well as the proton- and/or ion-gradient across the liposomal membrane are optimized to such magnitude that the liposomal uptake of solubility enhancing agent is essentially the same as the uptake of the sparingly water-soluble agent (e.g., the agent is taken up by the liposome in the form of a complex with the complexing agent). Preferably, the concentration of solubility enhancing agent in the internal aqueous medium of the loaded liposome is substantially less than that external to the liposome. In an exemplary embodiment, 5% or less of the concentration of solubility enhancing agent on the outside of the liposome is encapsulated in the internal aqueous medium of the liposome. The ratio of the solubility enhancing agent to the sparingly water-soluble agent, after the sparingly soluble agent is loaded into the liposome is greater than 200:1, e.g., 100:1, 60:1, 30:1, 10:1 etc.,.

As will be apparent from the foregoing, the rate and efficiency of active-loading a given sparingly water-soluble agent into the liposome is affected by many factors, especially by the transmembrane gradient, the choice of solubility enhancing agent, the composition of the liposome membrane, the process temperature, etc. It is within the capabilities and the normal routine of those skilled in the art to adapt and optimize these parameters in conjunction to arrive at the most efficient process for a given sparingly water-soluble agent.

In various embodiments, the use of a solubility enhancing agent as described in the foregoing in the active-loading of liposomes to enhance the loading efficiency and/or rate of sparingly water-soluble agents. In various embodiments the solubility enhancing agent is a complexing agent. As will be understood, exemplary embodiments involve combining the pre-formed liposomes, sparingly water-soluble agents, internal aqueous medium, external aqueous medium, gradients, etc. as defined in any of the foregoing. In an exemplary embodiment of the invention, the method includes combining the solubility enhancing agent with the sparingly water-soluble agent in a first aqueous medium (i.e. the external medium defined hereinbefore) and contacting the resulting complex with liposomes encapsulating a second aqueous medium (i.e., the internal medium) under conditions appropriate for the complex to be transferred across the membrane and encapsulated essentially intact in the aqueous compartment.

In a preferred embodiment of the invention, this composition has a sparingly-water-soluble-agent-to-lipid mass ratio of at least about 50:1, e.g., at least about 10:1, e.g., at least about 3:1, e.g., at least about 1:1, e.g., at least about 1:3, e.g., at least about 1:10.

Typically, the liposomal pharmaceutical formulation comprises the sparingly water-soluble agents mainly in the form of a liposome encapsulated agent and the agent in the complex with the solubility enhancing agent. In an exemplary embodiment, the agent complex constitutes less than ½ of the sparingly soluble drug in the formulation. In an exemplary embodiment, about 90% or greater of the agent is encapsulated in the aqueous compartment of the liposome and about 10% of the agent is in a complex with the solubility enhancing agent located external to the liposome

Furthermore, in an exemplary embodiment, the amount of solubility enhancing agent in the internal aqueous medium of the agent loaded liposomes is significantly less than the ratio of agent:solubility enhancing agent in the complex prior to its loading into the liposome. In various embodiments, the stoichiometric ratio of solubility enhancing agent:agent in the aqueous compartment of the liposome is not more than about 5 mol %, e.g., not more than about 3 mol %, e.g., not more than about 1 mol %, e.g., not more than about 0.1 mol %, e.g., not more than about 0.01 mol % of the ratio in the complex prior to encapsulation of the sparingly soluble drug in the aqueous compartment of the liposome.

In an exemplary embodiment, the invention utilizes a method in which a sparingly soluble agent with a protonatable amine in a CD complex is mixed with a preformed liposome containing an amine salt of an anionic compound. The mixture is incubated until the sparingly water-soluble agent released from the complex is concentrated in the internal aqueous medium of the liposome. The resulting aqueous core agent-loaded liposome is, in one embodiment, further processed to remove the CD. In an exemplary embodiment, the pharmaceutical formulation of aqueous core agent-loaded liposome and CD agent complex is administered to the patient with minimal (e.g., dilution, pH adjustment, osmolality or osmolarity adjustment, and/or filtration or other sterilization process) or no further processing following the preparation of the liposomal formulation.

In one embodiment in which the liposome formulation is to be administered by intramuscular or subcutaneous injection, the liposomes are large multivesicular (LMV) liposomes. LMV are prepared by (a) hydrating a lipid film with an aqueous solution containing an amine salt of an anionic molecule, such as a solution of ammonium sulfate (e.g., about 250 mM), (b) homogenizing the resulting suspension to form a suspension of small unilamellar vesicles (SUV), and (c) freeze-thawing said suspension of SUV at about −20° C. repeating the freeze thaw cycle at least three times. The extraliposomal ammonium sulfate is then removed, e.g. by dialysis against about 0.15 M NaCl or about 300 mM sucrose. The LMV liposomes are then mixed with a solution of the complex or used to rehydrate a lyophilized vial of the complex. Preferably, the complex contains a weakly basic moiety, and the suspension of LMV liposomes has a greater concentration of ammonium ions inside the liposomes than outside the liposomes. In an alternative implementation of this embodiment, the LMV is replaced by a multilamellar vesicle (MLV), e.g., with a particle diameter from about 0.5 to about 40 microns.

In another embodiment in which the liposome formulation is to be administered intravenously or intra-arterially, large unilamellar vesicles (LUV) are prepared by injection of a lipid solution in ethanol into an aqueous solution containing an amine salt of an anionic molecule, such as a solution of ammonium sulfate (e.g., about 250 mM) so that the concentration of ethanol is less than 30 v/v %. The resulting lipid dispersion is then extruded through polycarbonate membranes with a defined pore diameter of 100 nanometers (nm). The ethanol and non-entrapped ammonium sulfate is removed from the LUV suspension by dialysis in a dialysis cell against 300 mM sucrose 5 mM Tris buffer. The LUV which have a diameter of approximately 100 nm are then mixed with a solution of the complex or used to rehydrate a lyophilized vial of the complex. As with the LMV or MLV the agent contains a weakly basic moiety, and the suspension of LUV liposomes has a greater concentration of ammonium ions inside the liposomes than outside the liposomes.

The liposomes in suspension can be added to an aqueous solution of the CD-drug complex, a dried powder of the CD-drug complex or a lyophilized suspension of the CD-drug complex. Alternatively, the CD-drug complex in an aqueous solution can be added to liposomes containing an amine salt of an anionic molecule. The liposomes can be in aqueous suspension, or as dried powder of liposomes or as a lyophilized liposome.

In another embodiment the concentration of liposomes mixed with the CD-drug complex can be adjusted so that only a portion of the drug is loaded into the aqueous core of the liposome while the reminder of the drug is complexed with the CD and intercalated into the bilayer. When the mixture is administered into a patient there is a rapid drug release phase as the drug dissociates from the CD and bilayer and a slower drug release phase as the drug come out of the aqueous core. This allows a programmed drug release profile that provides a rapid drug level and a sustained drug level. Administering core drug-loaded liposomes with CD-drug complexes is another distinct advantage provided by this invention.

### Kits

In an exemplary embodiment, the invention provides a kit containing one or more components of the liposomes or formulations of the invention and instructions on how to combine and use the components and the formulation resulting from the combination. In various embodiments, the kit includes a complex formed between the sparingly water-soluble agent in one vessel and a liposome preparation in another vessel. Also included are instructions for combining the contents of the vessels to produce a liposome or a formulation thereof of the invention. In various embodiments, the amount of complex and liposome are sufficient to formulate a unit dosage formulation of the complexed agent.

In an exemplary embodiment, one vessel includes a liposome or liposome solution, which is used to convert at least part of the contents of a vessel of a lyophilized formulation of a polyanionic beta-cyclodextrin complexed with a therapeutic agent (e.g., an approved therapeutic agent) into a liquid formulation of the liposome encapsulated drug at the point of care for administration to a subject. In an exemplary embodiment, the contents of the vessels are sufficient to formulate a unit dosage formulation of the agent.

In the embodiment in which a unit dosage format is formed, the vessel includes from about 1 mg to about 500 mg of the approved agent, e.g, from about 1 mg to about 200 mg, e.g., from about 5 mg to about 100 mg, e.g., from about 10 mg to about 60 mg.

In an exemplary embodiment, the approved agent is carfilzomib and it is present in the vessel in an amount of from about 40 mg to about 80 mg, e.g., from about 50 mg to about 70 mg. In an exemplary embodiment, the carfilzomib is present in about 60 mg.

### Methods of Treatment

In one aspect, the invention provides a method of treating a proliferative disorder, e.g., a cancer, in a subject, e.g., a human, the method comprising administering a composition that comprises a pharmaceutical formulation of the invention to a subject in an amount effective to treat the disorder, thereby treating the proliferative disorder.

In one embodiment, the pharmaceutical formulation is administered in combination with one or more additional anticancer agent, e.g., chemotherapeutic agent, e.g., a chemotherapeutic agent or combination of chemotherapeutic agents described herein, and radiation.

In one embodiment, the cancer is a cancer described herein. For example, the cancer can be a cancer of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer, lung adenocarcinoma and squamous cell cancer), genitourinary tract, e.g., ovary (including fallopian tube and peritoneal cancers), cervix, prostate, testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and soft tissue (e.g., Kaposi's sarcoma (e.g., AIDS related Kaposi's sarcoma), leiomyosarcoma, angiosarcoma, and histiocytoma).

In an exemplary embodiment, the cancer is multiple myeloma or a solid tumor. In one embodiment, the pharmaceutical formulation of the invention includes carfilzomib as the sparingly water-soluble therapeutic agent.

In one aspect, the disclosure features a method of treating a disease or disorder associated with inflammation, e.g., an allergic reaction or an autoimmune disease, in a subject, e.g., a human, the method comprises: administering a composition that comprises a Pharmaceutical formulation of the invention to a subject in an amount effective to treat the disorder, to thereby treat the disease or disorder associated with inflammation.

In one embodiment, the disease or disorder associated with inflammation is a disease or disorder described herein. For example, the disease or disorder associated with inflammation can be for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatitis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, scleroderma, psoriasis, and dermatosis with acute inflammatory components. In some embodiments, the autoimmune disease is an organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), or Grave's disease.

In another embodiment, a pharmaceutical formulation of the invention or method described herein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The pharmaceutical formulation of the invention, particle or composition may be used to treat chronic hepatitis infection, including hepatitis B and hepatitis C.

In one aspect, the disclosure features a method of treating cardiovascular disease, e.g., heart disease, in a subject, e.g., a human, the method comprising administering a a pharmaceutical formulation of the invention to a subject in an amount effective to treat the disorder, thereby treating the cardiovascular disease.

In one embodiment, cardiovascular disease is a disease or disorder described herein. For example, the cardiovascular disease may be cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using a pharmaceutical formulation of the inventions, particles, compositions and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. Yet other disorders that may be treated with pharmaceutical formulation of the invention, include restenosis, e.g., following coronary intervention, and disorders relating to an abnormal level of high density and low density cholesterol.

In one embodiment, the pharmaceutical formulation of the invention can be administered to a subject undergoing or who has undergone angioplasty. In one embodiment, the Pharmaceutical formulation of the invention, particle or composition is administered to a subject undergoing or who has undergone angioplasty with a stent placement. In some embodiments, the pharmaceutical formulation of the invention, particle or composition can be used as a strut of a stent or a coating for a stent.

In one aspect, the invention provides a method of treating a disease or disorder associated with the kidney, e.g., renal disorders, in a subject, e.g., a human, the method comprises: administering a pharmaceutical formulation of the invention to a subject in an amount effective to treat the disorder, thereby treating the disease or disorder associated with kidney disease.

In one embodiment, the disease or disorder associated with the kidney is a disease or disorder described herein. For example, the disease or disorder associated with the kidney can be for example, acute kidney failure, acute nephritic syndrome, analgesic nephropathy, atheroembolic renal disease, chronic kidney failure, chronic nephritis, congenital nephrotic syndrome, end-stage renal disease, good pasture syndrome, interstitial nephritis, kidney damage, kidney infection, kidney injury, kidney stones, lupus nephritis, membranoproliferative GN I, membranoproliferative GN II, membranous nephropathy, minimal change disease, necrotizing glomerulonephritis, nephroblastoma, nephrocalcinosis, nephrogenic diabetes insipidus, nephrosis (nephrotic syndrome), polycystic kidney disease, post-streptococcal GN, reflux nephropathy, renal artery embolism, renal artery stenosis, renal papillary necrosis, renal tubular acidosis type I, renal tubular acidosis type II, renal underperfusion, renal vein thrombosis.

In an exemplary embodiment, the invention provides a method of treating metal toxicity or metal overload. Examples of diseases or disorders associated with metal include iron overload disorders (e.g., thalassemia or sickle cell anemia), copper over load disorders (e.g., Wilson's disease), and radioisotope contamination (e.g., occurring subsequent to contamination with plutonium, uranium and other radioistopes).

An “effective amount” or “an amount effective” refers to an amount of the pharmaceutical formulation of the invention which is effective, upon single or multiple dose administrations to a subject, in treating a cell, or curing, alleviating, relieving or improving a symptom of a disorder. An effective amount of the composition may vary according to factors such as the disease state, age, sex, and weight of the individual, and the ability of the compound to elicit a desired response in the individual. An effective amount is also one in which any toxic or detrimental effects of the composition are outweighed by the therapeutically beneficial effects.

As used herein, the term “prevent” or “preventing” as used in the context of the administration of an agent to a subject, refers to subjecting the subject to a regimen, e.g., the administration of a pharmaceutical formulation of the invention such that the onset of at least one symptom of the disorder is delayed as compared to what would be seen in the absence of the regimen.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

As used herein, the term “treat” or “treating” a subject having a disorder refers to subjecting the subject to a regimen, e.g., the administration of a pharmaceutical formulation of the invention such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

The following non-limiting examples are offered to illustrate selected embodiments of the invention.

### EXAMPLES

### Example 1

### General Liposome Preparation

Prior to liposome formation, lipids are dissolved in chloroform, and chloroform is removed under reduced pressure using a rotary evaporator to form a thin lipid film on the sides of a glass flask. The lipid film is dried overnight under a high vacuum. The lipid film is rehydrated with a 250 mM solution of ammonium sulfate (ammonium sulfate buffer). The mixture of lipid and buffer is placed under a nitrogen atmosphere and the lipid film is rehydrated at 60° C. by agitating the closed flask on a vortex mixer or by placing it into a bath sonicator and sonicating the dispersion for 5 minutes at 60° C. The lipid dispersion is extruded through a 200 nm polycarbonate membrane eleven times and then through a 100 nm polycarbonate membrane eleven times at 60° C. The extruded liposomes are held at 60° C. for 15 min and then cooled to room temperature. Liposomes are dialyzed at 4° C. against 100 volume excess buffer (5 mM HEPES, 10% sucrose (WN) (sucrose buffer) the pH adjusted to an appropriate value for loading) for 18-24 hours. This creates a liposome where the concentration of the ammonium sulfate is greater on the inside than on the outside.

Alternatively, lipids were dissolved in ethanol at a concentration of 500 mM HSPC/Cholesterol/PEG-DSPE: 3/2/0.15 (591 mg/mL total lipid) at 65° C. and the 9 volumes of the trapping agent solution heated to 65° C. was added to the ethanol/lipid solution also at 65° C. and the mixture was vortexed and transferred to a 10 mL thermostatically controlled (65° C.) Lipex Extruder. The LUV were formed by extruding 10 times through polycarbonate membranes having 0.1 um pores. After extrusion the liposomes were cooled on ice. The transmembrane electrochemical gradient was formed by purification of the liposomes by dialysis in dialysis tubing having a molecular weight cut off of 12,000-14,000. The samples are dialyzed against 5 mM HEPES, 10% sucrose pH 6.5 (stirring at 4° C.) at volume that is 100 fold greater than the sample volume. The dialysate was changed after 2 h then 4 more times after 12 h each. The conductivity of the liposome solution was measured and was indistinguishable from the dialysis medium ˜400/cm.

In the case of liposomes with diameters less than 350 nm they are filtered through a 0.45 micron sterile filter into a sterile container. Multilamellar (MLV) or oligolameller (OLV) vesicles are prepared under aseptic conditions using pre-sterilized buffers (G. Gregoriadis, Ed., Liposome Technology, (2006) Liposome Preparation and Related Techniques, 3rd Ed.). Following their manufacture in ammonium sulfate buffer and dialysis against 100 volumes of sucrose buffer they are extruded through a 2 micron polycarbonate membrane into a sterile container. The usually total lipid concentration before dialysis of LUV is 20 mM and of MLV is 100 mM, unless otherwise indicated. Average liposome diameter and zeta potential are determined by dynamic light scattering measurements (Malvern Instruments Zetasizer Nano ZS). For liposomes extruded through the 100 nm polycarbonate membrane the liposome diameter is approximately 100 nm. For LMV, MLV or OLV liposomes the diameters can range from 0.5 microns to 40 microns before extrusion after 0.5 to 3 microns extrusion through the 2 micron polycarbonate membrane depending upon the preparation.

The lipid concentration is determined by measuring the cholesterol by HPLC using an Agilent 1100 HPLC with and Agilent Zorbax 5 um, 4.6×150 mM, Eclipse XDB-C8 column and a mobile phase of A=0.1% TFA, B=0.1% TFA/MeOH with an isocratic elution of 99% B. The flow rate is 1.0 mL/min, column temperature is 50° C., 10 μL injection and detection by absorbance at 205 nm. The lipid concentration is determined by measuring the cholesterol by HPLC using an Agilent 1100 HPLC with and Agilent Zorbax 5 um, 4.6×150 mM, Eclipse XDB-C8 column and a mobile phase of A=0.1% TFA, B=0.1% TFA/MeOH with an isocratic elution of 99% B. The flow rate is 1.0 mL/min, column temperature is 50° C., 10 μL injection and detection by absorbance at 205 nm. The retention time of cholesterol is 4.5 min.

The HPLC analysis of carfilzomib and amioderone was performed on the same system as described for analysis of cholesterol. The mobile phase consists of A=0.1% TFA, B=0.1% TFA/MeOH with a gradient elution starting at 50% B and increasing to 83% B in 13 min with 7 min equilibration back to 50% B. The flow rate is 1.0 mL/min, column temperature is 30 C, 10 μL injection and detection by absorbance at 205 nm. The retention time of carfilzomib is 12.2 min and amioderone was 13.2 min.

The HPLC analysis of all other drugs used (such as ziprasidone, ariprazole, voriconazole etc) was performed on the same system as described for analysis of cholesterol. The mobile phase consists of −A=0.1% TFA, B=0.1% TFA/MeOH with a gradient elution starting at 40% B and increasing to 70% B in 7 min with 6 min equilibration back to 40% B. The flow rate is 1.0 mL/min, column temperature is 30 C, 10 μL injection and detection by absorbance at 254 nm. The retention times were as follows: ziprasidone 5.73 min, ariprazole 8.12 min, voriconazole 8.81 min.

The liposome diameter is measured by dynamic light scattering on a Malvern zeta sizer.

### Example 2

### Conversion of Geodon® (Ziprasidone Hydrochloride) from a Sulfobutylether β-Cyclodextrin Sodium (SBCD) Formulation into a LUV Liposome Ziprasidone SBCD Formulation

Ziprasidone is an atypical antipsychotic available as a lyophilized cake that after reconstitution can be administered as an intramuscular injection. GEODON® is available in a single-dose vial as ziprasidone mesylate (20 mg of ziprasidone and 4.7 mg of methanesulfonic acid solubilized by 294 mg of sulfobutylether β-cyclodextrin sodium (SBCD).

A portion of the lyophilized cake of GEODON® (Pfizer) was weighed out and dissolved in deionized water. The amount of the active ingredient (ziprasidone) was calculated by multiplying the cake weight by 0.0625 and added to liposomes from a 1 mg/mL solution. In certain experiments the lyophilized cake was reconstituted with the aqueous liposome preparation at defined lipid to drug ratios (liquid/liquid system). In other cases the lyophilized cake was directly rehydrated with the liposome suspension (liquid/solid system). The LUV were diluted with 5 mM Hepes, 10% sucrose pH 6.5. The liposome/drug solution was transferred to a 65° C. bath and swirled every 30 s for the first 3 min and then swirled every 5 min over a total heating time of 30 min. After heating for 30 min all samples were placed on ice for 15 min. the loaded liposomes were vortexed and 100 μL of sample was kept as the “before column” and the rest purified by ion exchange chromatography and gel filtration chromatography. Amberlite IRA-67 anion exchange resin was prepared by washing with 1N NaOH, followed by 3 washes with ddH₂O and 3 washes with 3N HCl. The resin was exhaustively washed with dH₂O until the pH of the supernatant was neutral. The SBCD was removed by adding 70 mg of resin to the eppendorf containing the liposomes and mixed well. The LUV solution was then purified by gel filtration chromatography (Sephadex G25) to remove residual extraliposomal drug. The turbid liposome fraction was collected and analyzed by HPLC.

In Table 1 are the results obtained when the Geodon powder was reconstituted with LUV (liquid/solid system) to obtain drug to lipid ratios (gram drug/mole total lipids) of 50, 100, 200 and 400. The percent encapsulation was 90.4, 75.5, 62.7 and 67.4% respectively. Thus at the lower drug to lipid ratios 90% of the drug was encapsulated. This data documents that the amount of liposomes added to the dry powder allows when to adjust the ratio of the drug in SBCD complex to amount of drug stably encapsulated in the liposomes from 1/9 to about 4/6.

We compared the effect on the loading efficiency between first rehydrating the Geodon powder and mixing it with liposomes in solution (liquid/liquid system) to directly rehydrating the lyophilized powder with an LUV suspension (liquid solid system). This data is also indicated in Table 1. At the 50 ratio there was no difference in the two methods. As the drug to lipid ratio was increased to 100 and then to 200 the loading from the liquid/liquid system was slightly better than from the solid/liquid system (at 200 D/L ratio, 67.4% liquid/solid versus 80.4% liquid/liquid).

TABLE 1
Encapsulation of Ziprasidone into liposomes

| Input | % ziprasidone entrapped in | % ziprasidone entrapped in |
|---|---|---|
| drug-to-lipid | liposome from reconstitution | liposome from suspension |
| (ug/umol) | of Geodon ™ cake | of ziprasidone |
| 50 | 90.42 ± 2.91 | 87.92 ± 2.35 |
| 100 | 75.50 ± 2.91 | 84.24 ± 3.14 |
| 200 | 62.68 ± 3.27 | 80.38 ± 3.61 |
| 400 | 67.40 ± 3.88 | N.D |
| 800 | 41.38 ± 0.81 | N.D |

### Example 3

### Conversion of Geodon® (Ziprasidone Hydrochloride) from a Sulfobutylether β-Cyclodextrin Sodium (SBCD) Formulation into a MLV Liposome Ziprasidone SBCD Formulation

Larger diameter liposomes are preferred in formulations that need to be retained at the site of injection. A vial of GEODON® containing 20 mg of ziprasidone and 4.7 mg of methanesulfonic acid and 294 mg of SBCD is aseptically reconstituted by slowly injecting 1.2 mL of MLV (100 mM total lipid, composition POPC/Chol/DSPG Mole ratio 3/2/0.15) loaded with ammonium sulfate and suspended in sucrose buffer The mixture is gently swirled or the vial is inverted slowly for about until complete dissolution of any cake or powder occurs. The mixture is allowed to stand at 65° C. for 60 minutes. At this time the preparation is transferred to an eppendorf centrifuge tube and subsequently centrifuged at 18,000 RPM for 10 minutes. The supernatant (0.9 mL) is removed and the pellet which contains sedimented liposomes is resuspended in 0.9 mL sucrose buffer. The mixture is subsequently centrifuged again at 18,000 RPM for 10 minutes. Then 0.9 mL of the clear supernatant is removed and the pellet is resuspended in 0.9 mL sucrose buffer A sample is lysed, an aliquot is injected into a HPLC system and the concentration of ziprasidone in the injected sample is determined. The percent of ziprasidone encapsulated in the MLV depends upon the drug/lipid ratio. The material is suitable for injection I.M. to provide a rapid release of ziprasidone from the SBCD complex as well as a sustained release of ziprasidone from inside of the MLV.

### Example 4

### Conversion of Ability® (Aripiprazole) from a SBCD Complex in Solution into a Liposome Encapsulated Aripiprazole/SBCD Complexed Ariprazole Mixture

ABILIFY® Injection is available in single-dose vials as a ready-to-use, 9.75 mg/1.3 mL (7.5 mg/mL) clear, colorless, sterile, aqueous solution for intramuscular use only. Inactive ingredients for this solution include 150 mg/mL of sulfobutylether β-cyclodextrin (SBECD), tartaric acid, sodium hydroxide, and water for injection. The active ingredient in Abilify® (Bristol-Myers), aripiprazole (7.5 mg/mL) was introduced to the LUV (HSPC/Cholesterol/PEG-DSPE: 3/2/0.15 mole ratio) at a drug to total lipid ratio of 50 g drug/mol HSPC (drug to total lipid ratio (wt/wt) of 0.12) or 100 g/mol (0.24 wt/wt) or 200 g/mol (0.48 wt/wt). The protocol used to encapsulate and characterize the product of ziprasidone liposomes in example 1 was also followed to encapsulate ariprazole and analyze the aripiprazole LUV in example 3. The percent encapsulation of 68.2, 48.4 and 43.2 at the drug/lipid ratios of 50, 100 and 200 respectively (Table 2).

TABLE 2
Encapsulation of ariprazole in LUV in a liquid/liquid protocol

| Input drug-to-lipid | % aripiprazole entrapped in liposome |
|---|---|
| (ug/umol) | when transferred from Abilify ™ |
| 50 | 60.16 ± 3.57 |
| 100 | 48.36 ± 3.78 |
| 200 | 43.22 ± 2.09 |

### Example 5

### Preparation of an SBCD-Aripiprazole Complex into a Mixture of SBCD-Aripiprazole and Liposome Encapsulated Aripiprazole by Reconstitution of a Lyophilized Liposome Preparation Containing Encapsulated Ammonium Sulfate with the Contents of an ABILIFY® Vial

Larger diameter liposomes are preferred in formulations that need to be retained at the site of injection. MLV composition POPC/Chol/DSPG Mole ratio 3/2/0.15 at 50 μmoles/mL total lipid are loaded with 250 mM ammonium sulfate. The preparation is then dialyzed against 5 mM Hepes, 10% sucrose pH 6.5 buffer to remove ammonium sulfate from the outside. Two mL of the MLV suspension are lyophilized to form a dry MLV cake. The liposome cake is gently rehydrated with the 1.3 mL contents of the ABILIFY® vial. The mixture is gently swirled or the vial is inverted slowly for about until complete dissolution of any cake or powder occurs. The mixture is allowed to stand at room temperature for 60 minutes. The percent encapsulation of the aripiprazole drug is measured by transferring the mixture to an eppendorf centrifuge tube and subsequently centrifuged at 18,000 RPM for 10 minutes. The supernatant (approximately 0.9 mL) is removed and the pellet which contains sedimented liposomes is resuspended in 0.9 mL sucrose buffer. The mixture is subsequently centrifuged again at 18,000 RPM for 10 minutes. Then 0.9 mL of the clear supernatant is removed and the pellet is resuspended in 0.9 mL sucrose buffer. A sample is lysed, an aliquot is injected into a HPLC system and the concentration of aripiprazole in the injected sample is determined. The percent of aripiprazole encapsulated in the MLV depends upon the drug/lipid ratio. The mixture of aripiprazole SBCD and liposome aripiprazole is suitable for injection I.M. to provide both a rapid release of aripiprazole from the SBCD complex as well as a sustained release of aripiprazole from inside of the MLV.

### Example 6

### Conversion of a Lyophilized MLV Formulation with Encapsulated Triethylamine Sulfate into a Liposome Cerenia® (Maropitant Citrate) SBCD Liquid Formulation

Maropitant citrate is used for the prevention of acute vomiting in dogs and cats. The MLV formulation described in example 4 is prepared with 250 mM triethylamine sulfate in place of ammonium sulfate. The formulation is lyophilized to provide a cake consisting of 120 mg sucrose and a 1.2 μmoles total lipid of a liposome with a lipid composition of POPC/Chol/DSPG mole ratio 3/2/0.15 with a higher concentration of triethylamine sulfate on the inside than on the outside. The contents of the 20 mL Cerenia® Injectable Solution vial which contains 10 mg maropitant, 63 mg sulphobutylether-beta-cyclodextrin, 3.3 mg meta-cresol per mL is slowly added to the vial of lyophilized liposomes and the vial is slowly rotated to rehydrate the MLV at room temperature. The milky white mixture is allowed to stand at 60° C. for 30 minutes. The percent of the maropitant that is incorporated into the liposome is about 60% of the total maropitant in the vial. This results in a drug to lipid ratio of about 140 grams/mole lipid. Approximately 40% of the drug remains associated with the SBCD. This formulation is suitable for subcutaneous injection to prevent vomiting associated with chemotherapy.

### Example 7

### Loading of Carfilzomib from the Lyophilized Vial into the Core of an Added LUV

Carfilzomib is an anticancer drug that is soluble at less than 10 micrograms/mL in water. The drug is formulated as a sulfobutylether beta-cyclodextrin complex with the brand name of Kyprolis®. Carfilzomib undergoes rapid hydrolysis in water so it is formulated as a sterile lyophilized cake. The vial contains 60 mg (0.08 μmoles) of carfilzomib, 3000 mg sulfobutylether beta-cyclodextrin (1.4 μmoles) so the CD to drug ratio is 16/1. The vial also contains 57.7 mg citric acid, and sodium hydroxide for pH adjustment to about pH 3.5.

A 30 mL preparation of LUVs composed of HSPC/Cholesterol/PEG2000-DSPE at lipid concentration of 15 mM lipid (circa 300 mg lipid) is prepared by the extrusion process through 100 nm polycarbonate membranes in a 250 mM ammonium sulfate solution to yield a 100 nm diameter LUV suspension of encapsulated ammonium sulfate. The LUV are dialyzed against 500 volumes of 0.15 M NaCl to remove non-encapsulated ammonium sulfate. The liposome suspension is then passed through a 0.2 micron sterile membrane into a sterile tube. The liposome solution (29 mL) is added aseptically to the vial containing 60 mg of carfilzomib at room temperature. The vial is gently mixed not shaken, for about 1 minute, or until the cake or powder is completely solubilized. The rehydrated solid is incubated for 40 minutes at 37° C. The carfilzomib is substantially loaded (>60%) into the core of the liposome at the end of the incubation period. The 100 nm diameter of the liposomes is not altered by this rehydration incubation process. The temperature of the vial is then reduced to 4° C. until the mixture of CD, drug-loaded liposome are injected into the subject. This preparation will greatly increased the half-life of carfilzomib in blood and enable a greater fraction of the injected dose to deposit into the tumor as is found with other liposomal drugs were the drug is in the aqueous core of the liposome.

An additional non-obvious advantage of this formulation is that the liposome encapsulated carfilzomib is more stable in aqueous solution than the currently used sulfobutylether beta-cyclodextrin carfilzomib complex.

After learning of this protocol, it would be apparent to one who is familiar with the field that other liposome compositions containing triethylamine sulfate or other amine anions could be used to rehydrate the carfilzomib.

### Example 8

### Partial Loading of Carfilzomib from the Lyophilized Vial into the Core of an Added LUV

The pharmacokinetic behavior of preparation of carfilzomib can be easily modulated by adjusting the amount of LUV containing ammonium sulfate or triethylamine sulfate that are added to the vial of Kyprolis®. If the amount of liposomes added to the vial in example 1 is decreased from 300 mg to 200 mg of total lipid the amount of carfilzomib that remains complexed to the CD will be about one-half of the total amount in the carfilzomib vial. When this formulation is injected into a subject about one-half of the carfilzomib will be rapidly availability to interact with the target site and the remainder would be slowly distributed into the body and the tumor. This allows formulation approach will allow the optimal dose pharmacokinetic profile to be identified in clinical trials using a simple and reproducible protocol. The formulation would be in two or three vials: the first containing the lyophilized carfilzomib-CD complex. The second containing the sterile ammonium sulfate loaded liposome suspension in 0.15 M NaCl and if necessary the third vial containing a buffer to adjust the pH of the final preparation so the mixture can be administered into patients.

This formulation approach will allow the optimal dose or pharmacokinetic profile to be identified in clinical trials using a simple and reproducible protocol. The formulation is in two vials: the first vial containing the lyophilized carfilzomib-CD complex that is the current Kyprolis®. The second vial containing the sterile triethylamine sulfate loaded liposome suspension in 10% sucrose-5 mM Hepes, pH7.4 buffer. Reconstitution of the Kyprolis® with the contents of the second vial can be done in the pharmacy.

Vials can be prepared with different liposome compositions, diameters, targeting ligands on the liposome surface or amine salt compositions. This would enable the rapid screening in early phase clinical trials of carfilzomib liposome SBCD mixtures to identify the formulations that provide optimized pharmacokinetic and biodistribution profiles of carfilzomib for follow up clinical trials.

### Example 9

### Pharmacokinetics in Mice of SBCD Complexed Carfilzomib (a) or a Mixture of SBCD-Carfilzomib and LUV Encapsulated Carfilzomib (b)

To illustrate the ability a mixture of SBCD complexed carfilzomib mixed with LUV encapsulated carfilzomib to increase the half-life of the carfilzomib in the blood of mice, a pharmacokinetic study is done in two groups of three Balb/c mice each. A 20 mg/kg dose of each of the two formulations is injected into the respective group of mice. Carfilzomib in SBCD is reconstituted from the Kyprolis® vial (The vial contains 60 mg (0.08 μmoles) of carfilzomib, 3000 mg sulfobutylether beta-cyclodextrin (1.4 μmoles) so the CD to drug ratio is 16/1 at a pH=3.5. The vial is reconstituted with 29 mL of sterile water. A sample of 0.20 mL is injected via the tail vein of each mouse in the SBCD-carfilzomib group.

A vial of Kyprolis® is reconstituted with 29 mL of LUV containing ammonium sulfate as described in example 6. A 0.2 mL of the mixture of the SBCD carfilzomib and liposome encapsulated carfilzomib is injected into the tail vein of each mouse in the group. At 300 minutes post-injection, the animals are sacrificed and blood samples acquired from each animal.

The samples are processed and the amount of carfilzomib in the plasma determined on a HPLC assay. The low end for reasonably accurate detection using the HPLC is 2 microg/mL in the vial. The plasma is diluted 5 fold for drug extraction. When the 0.2 mL of a 2 mg/mL is injected into a mouse with 0.8 mL mouse plasma, the t=0 mouse plasma concentration is 500 microg/mL. Samples from animals that receive the SBCD carfilzomib complex are below the limit of detection of the HPLC assay. Samples that receive the mixture of the SBCD carfilzomib and LUV carfilzomib are detected at a concentration greater than 30 microg/mL. This is attributed primarily to the carfilzomib that remains encapsulated in the LUV in the plasma.

### Example 10

### Example Loading of CD Complexed 7-Ethyl-10-Hydroxy-Camptothecin (SN-38) into LUV Liposomes Containing the Acetate Salt of Low Molecular Weight Polyethyleneimine

SN-38, the active metabolite of camptothecin, is sparingly soluble. SN-38 can be solubilized by forming the hydroxypropyl beta cyclodextrin complex or by incorporating it into the liposomes bilayer. In either case, SN-38 rapidly transfers from the carrier when the formulation is injected into humans; successful tumor targeting does not occur for SN-38 delivered in either of these formulations.

Lipids are dissolved in ethanol at a concentration of 500 mM POPC/Cholesterol/PEG-DSPE: 3/2/0.15 (591 mg/mL total lipid) at 65° C. and the 9 volumes of the trapping agent solution, polyethyleneimine (PEI) MW 1300 titrated to pH 7.4 with glacial acetic acid to form the acetate polyethyleneimine salt, is heated to 65° C. then added to the ethanol/lipid solution also at 65° C. This mixture is vortexed and transferred to a 10 mL thermostatically controlled (65° C.) Lipex Extruder. The LUV are formed by extruding 10 times through polycarbonate membranes having 0.1 um pores. After extrusion, the LUV are cooled on ice. A transmembrane electrochemical gradient is formed by placing the liposomes in dialysis tubing having a molecular weight cut off of 12,000-14,000. The samples are dialyzed against 5 mM HEPES, 10% sucrose pH 6.5 (stirring at 4° C.) at volume that is 100-fold greater than the liposome sample volume. The dialysate is changed after 2 h then 4 more times after 12 h each. The conductivity of the liposome solution was measured and was indistinguishable from the dialysis medium ˜40 μS/cm.

The LUV (20 mM total lipid containing a greater concentration of acetate-PEI salt on the inside than on the outside) are mixed with the hydroxypropyl cyclodextrin solubilized SN-38 (24 mg/mL hydroxylpropylcyclodextrin and 2 mg/mL SN-38) and allowed to incubate for 12 hours at room temperature. The SN-38 is transferred into the liposome to approximately 50% of the added SN-38. The amount transferred can be adjusted from 10% to 90% by changing the initial ratio of the CD-SN-38 to LUV ratio or by adjusting the length of time the incubation is allowed to proceed.

### Example 11

### Loading Nexterone® (Amiodarone HCl) into Liposomes

Each mL of Nexterone® contains: 1.8 mg amiodarone HCl, 18 mg sulfobutylether β-cyclodextrin sodium, 0.362 mg citric acid anhydrous, 0.183 mg sodium citrate dihydrate, and 41.4 mg dextrose anhydrous in water for injection. Sodium hydroxide or hydrochloric acid may have been added to adjust pH. Amiodarone tightly binds to saturated phospholipids so it is best formulated in a liposome composed of a sterol modified such as OleoylChems Phosphatidylcholine (OChemsPC-, obtained Avanti Polar Lipids, inc.) to reduce the membrane disruptive properties of the drug. LUV composed of OChemsPC and PEG-DSPE: 95/5 at 30 mM total concentration are prepared, loaded a higher-ammonium sulfate concentration on the inside than on the outside as described in Example 2. Ten mL of the OChemsPC liposomes are lyophilized to form a dry cake.

Ten mL of the Nexterone®-SBCD complex is used to rehydrate the OChemsPC liposomes. The mixture is gently swirled or the vial is inverted slowly for about until complete dissolution of any cake or powder occurs. The mixture is allowed to stand at room temperature for 30 minutes. At this time the preparation is processed as described in Example 2. The percent of amiodarone encapsulated in the LUV depends upon the drug/lipid ratio and time of incubation. The material is suitable for injection I.V. to provide a rapid release of amiodarone from the SBCD complex as well as a sustained release of amiodarone from inside of the LUV.

### REFERENCES

S. M. Berge et al., J. Pharm. Sci., (1977) 66:1-19.
R. Challa et al., AAPS PharmSciTech, (2005) 6(2) E329-E357.
P. R. Cullis et al., Biochimica et Biophysica Acta, (1997) 1331:187-211.
D. Drummond et al., J. Pharm. Sci., (2008) 97(11):4696-4740.
G. Gregoriadis, Ed., Liposome Technology, (1984) Vol. I, CRC Press, Boca Raton, Fla.
G. Gregoriadis, Ed., Liposome Technology, (2006) Liposome Preparation and Related Techniques, 3rd Ed.
T. Loftsson, M. E. Brewster, Pharmaceutical Applications of Cyclodextrins: Basic Science and Product Deveopment, J. Pharm. Pharmacol. (2010) 62(11):1607-1621.
D. Zucker et al., Journal of Controlled Release (2009) 139:73-80.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the claims appended hereto and their equivalents.

All publications, patents, and patent applications cited herein are hereby incorporated by reference in their entirety for all purposes.

What is claimed is:

1. A pharmaceutical formulation of a sparingly water-soluble therapeutic agent, said formulation comprising:
(a) a pharmaceutically acceptable diluent;
(b) a complex between said sparingly water-soluble therapeutic agent and a solubility enhancing agent dissolved in said diluent; and
(c) a population of liposomal lipid vesicles suspended in said diluent, wherein said lipid vesicles comprise a lipid membrane encapsulating an internal aqueous medium comprising a first fraction of said sparingly water-soluble therapeutic agent, which is not complexed with a solubility enhancing agent.

2. The pharmaceutical formulation according to claim 1, wherein the solubility enhancing agent is a complexing agent selected from the group of cyclodextrins and their derivatives, povidones, and combinations thereof.

3. The pharmaceutical formulation according to claim 1, wherein the solubility enhancing agent is not an alcohol that permeabilizes the liposomal membrane.

4. The liposome according to claim 1, wherein said solubility enhancing agent is a member selected from sulfobutylether beta-cyclodextrin or hydroxypropylether beta-cyclodextrin.

5. The pharmaceutical formulation according to claim 1, wherein said therapeutic agent is a small organic molecule having a water-solubility of less than or equal to about 2 mg/mL.

6. The pharmaceutical formulation according to claim 1, wherein the therapeutic agent is a member selected from an anthracycline compound, a camptothecin compound, a vinca alkaloid, an ellipticine compound, a taxane compound, a wortmannin compound, a geldanamycin compound, a pyrazolopyrimidine compound, a steroid compound, a peptide-based compound, a derivative of any of the foregoing, a pro-drug of any of the foregoing, and an analog of any of the foregoing.

7. The pharmaceutical formulation according to claim 1, wherein said therapeutic agent is selected from voriconazole, amiodarone, ziprasidone, aripiprazole, carfilzomib, imatinib, lapatinib, oprozomib, cyclopamine, CUR-61414, PF-05212384, PF-4691502, toceranib, PF-477736, PF-337210, sunitinib, SU14813, axitinib, AG014699, veliparib, MK-4827, ABT-263, SU11274, PHA665752, Crizotinib, XL880, PF-04217903, XR5000, AG14361, veliparib, bosutunib, PD-0332991, PF-01367338, AG14361, NVP-ADW742, NVP-AUY922, NVP-LAQ824, NVP-TAE684, NVP-LBH589, erubulin, doxorubicin, daunorubicin, mitomycin C, epirubicin, pirarubicin, rubidomycin, carcinomycin, N-acetyladriamycin, rubidazone, 5-imido daunomycin, N-acetyl daunomycin, daunoryline, mitoxanthrone, camptothecin, 9-aminocamptothecin, 7-ethylcamptothecin, 7-ethyl-10-hydroxy-camptothecin, 10-hydroxycamptothecin, 9-nitrocamptothecin,1O,11-methylenedioxycamptothecin, 9-amino-1O,11-methylenedioxycamptothecin, 9-chloro-10,11-methylenedioxycamptothecin, irinotecan, lurtotecan, silatecan, (7-(4-methylpiperazinomethylene)-10,11-ethylenedioxy-20(S)-camptothecin, 7-(4-methylpiperazinomethylene)-10, II-methylenedioxy-20(S)-camptothecin, 7-(2-N-isopropylamino)ethyl)-(20S)-camptothecin, CKD-602, vincristine, vinblastine, vinorelbine, vinflunine, vinpocetine, vindesine, ellipticine, 6-3-aminopropyl-ellipticine, 2-diethylaminoethyl-ellipticinium, datelliptium, retelliptine, paclitaxel, docetaxel, diclofenac, bupivacaine, 17-Dimethylaminoethylamino-17-demethoxygeldanamycin, cetirizine, fexofenadine, Onx 0912, Onx 0914, PD0332991, Axitinib, Lenvatinib,PHA665752, SU11274, PF-02341066, foretinib, XL880, PX-478, GDC-0349, PD0332991, AZD4547, Golotimod, SCH900776, TG02, UNCO638, ARRY-520, Elacridar hydrochloride, golvatinib, MK-1775, PF-03758309, AT13387, BAY 80-6946, cobicistat, GDC-0068, INNO-206, MLN0905, resminostat, tariquidar, primidone and other catecholamines, epinephrine, salts, prodrugs and derivatives of these medicinal compounds and mixtures thereof.

8. The pharmaceutical formulation according to claim 1, wherein said therapeutic agent is selected from an antihistamine ethylenediamine derivative, bromphenifamine, diphenhydramine, an anti-protozoal drug, quinolone, iodoquinol, an amidine compound, pentamidine, an antihelmintic compound, pyrantel, an anti-schistosomal drug, oxaminiquine, an antifungal triazole derivative, fliconazole, itraconazole, ketoconazole, miconazole, an antimicrobial cephalosporin, thelating agents, deferoxamine, deferasirox, deferiprone, FBS0701, cefazolin, cefonicid, cefotaxime, ceftazimide, cefuoxime, an antimicrobial beta-lactam derivative, aztreopam, cefinetazole, cefoxitin, an antimicrobial of erythromycin group, erythromycin, azithromycin, clarithromycin, oleandomycin, a penicillin compound, benzylpenicillin, phenoxymethylpenicillin, cloxacillin, methicillin, nafcillin, oxacillin, carbenicillin, a tetracycline compound, novobiocin, spectinomycin, vancomycin; an antimycobacterial drug, aminosalicycic acid, capreomycin, ethambutol, isoniazid, pyrazinamide, rifabutin, rifampin, clofazimine, an antiviral adamantane compound, amantadine, rimantadine, a quinidine compound, quinine, quinacrine, chloroquine, hydroxychloroquine, primaquine, amodiaquine, mefloquine, an antimicrobial, qionolone, ciprofloxacin, enoxacin, lomefloxacin, nalidixic acid, norfloxacin, ofloxacin, a sulfonamide; a urinary tract antimicrobial, nitrofurantoin, trimetoprim; anitroimidazoles derivative, metronidazole, a cholinergic quaternary ammonium compound, ambethinium, neostigmine, physostigmine, an anti-Alzheimer aminoacridine, tacrine, an anti-parkinsonal drug, benztropine, biperiden, procyclidine, trihexylhenidyl, an anti-muscarinic agent, atropine, hyoscyamine, scopolamine, propantheline, an adrenergic compound, dopamine, serotonin, a hedgehog inhibitor, albuterol, dobutamine, ephedrine, epinephrine, norepinephrine, isoproterenol, metaproperenol, salmetrol, terbutaline, a serotonin reuptake inhibitor, an ergotamine derivative, a myorelaxant, a curare series, a central action myorelaxant, baclophen, cyclobenzepine, dentrolene, nicotine, a nicotine receptor antagonist, a beta-adrenoblocker, acebutil, amiodarone, abenzodiazepine compound, ditiazem, an antiarrhythmic drug, diisopyramide, encaidine, a local anesthetic compound, procaine, procainamide, lidocaine, flecaimide, quinidine, an ACE inhibitor, captopril, enelaprilat, Hsp90 inhibitor, fosinoprol, quinapril, ramipril; an opiate derivative, codeine, meperidine, methadone, morphine, an antilipidemic, fluvastatin, gemfibrosil, an HMG-coA inhibitor, pravastatin, a hypotensive drug, clonidine, guanabenz, prazocin, guanethidine, granadril, hydralazine, a non-coronary vasodilator, dipyridamole, an acetylcholine esterase inhibitor, pilocarpine, an alkaloid, physostigmine, neostigmine, a derivative of any of the foregoing, a pro-drug of any of the foregoing, and ananalog of any of the foregoing.

9. The pharmaceutical formulation according to claim 1, wherein said lipid membrane comprises one or more member selected from egg phosphatidylcholine (EPC), egg phosphatidylglycerol (EPG), dipalmitoylphosphatidylcholine (DPPC), sphingomyelin (SM), cholesterol (Chol), cholesterol sulfate and its salts (CS), cholesterol hemisuccinate and its salts (Chems), cholesterol phosphate and its salts (CP), cholesterol phthalate, cholesterylphosphorylcholine, 3,6,9-trioxaoctan-I-oI-cholesteryl-3e-ol, dimyristoylphosphatidylglycerol (DMPG), dimyristoylphosphatidylglycerol (DMPG), dimyristoylphosphatidylcholine (DMPC), distearoylphosphatidylcholine (DSPC), hydrogenated soy phosphatidylcholine (HSPC), distearoylphosphatidylglycerol (DSPG), cationic lipids, sterol modified lipids (SML), or inverse-zwitterlipids.

10. The pharmaceutical formulation according to claim 1, further comprising a second fraction of said sparingly water-soluble agent partitioned within the lipid membrane, wherein at least a population of members of said second fraction are not complexed with said solubility enhancing agent.

11. The pharmaceutical formulation according to claim 1, wherein the encapsulated agent is released from the lipid vesicle at a rate different than the rate at which the agent is released from the complex.

12. The pharmaceutical formulation of claim 11, wherein the rate encapsulated agent is released from the lipid vesicle is slower than the rate at which the agent is released from the complex.

13. The pharmaceutical formulation of claim 11, wherein release of said agent is bimodal.

14. A method of treating a subject in need of such treatment with a therapeutic agent, said method comprising administering to said patient a therapeutically effective amount of a pharmaceutical formulation according to claim 1.

15. A method of preparing a pharmaceutical formulation according to claim 1, said method comprising:
a) incubating an aqueous mixture comprising:
i. liposomes having said liposomal lipid membrane encapsulating an internal aqueous medium;
ii. said complex between said solubility enhancing agent and said first fraction of said sparingly water-soluble therapeutic agent;
iii. an aqueous medium external to said liposomes in which said liposomes are suspended,
wherein a member selected from a proton gradient, an ion gradient and a combination thereof exists across said liposomal membrane between said internal aqueous medium and said aqueous medium external to said liposomes;
said incubating being for a period of time sufficient to cause at least part of sparingly water-soluble therapeutic agent in said complex to be drawn out of the aqueous medium external to said liposomes and to accumulate in said internal aqueous medium under the influence of a member selected from said proton gradient, said ion gradient and a combination thereof.

16. The method according to claim 15, wherein said sparingly water-soluble substance has a solubility in water of <1.9 mg/mL.

17. The method according to claim 15, wherein the solubility enhancing agent increases the initial concentration of the sparingly-water soluble substance in the external aqueous medium at least two-fold, to values of at least about 1.9 mg/mL at about 20° C.

18. The method according to claim 15, wherein a pH gradient, a sulfate gradient, a phosphate gradient, a citrate gradient an acetate gradient, an EDTA-ion gradient, an ammonium-salt gradient, an alkylated ammonium-salt gradient, a Mg²⁺, Mn²⁺-, Cu²⁺-, Na⁺-, K⁺-gradient, or a combination thereof exists across the liposomal membrane during step said incubating.

19. The method according to claim 15, wherein said sparingly water-soluble substance contains a member selected from protonizable amine, a carboxyl function and a combination thereof.

20. The method according to claim 15, wherein step said incubating is for a period of time sufficient to achieve a loading into said internal aqueous medium of at least 25% of the total amount of said complex in said external aqueous medium.

21. A method of preparing a pharmaceutical formulation according to claim 1, said method comprising: contacting a dry or lyophilized fraction of the complexed therapeutic agent with an aqueous suspension of said liposome under conditions appropriate for encapsulation of said uncomplexed agent within said internal aqueous core of said liposome.

22. The method according to claim 21, wherein said conditions comprise a gradient of pH or charge across said vesicle membrane.

23. A kit comprising: a vessel containing a cyclodextrin complexed sparingly water-soluble therapeutic agent; a vessel containing a liposome suspension with an ion gradient such that the ion concentration is higher inside of said liposome than outside said liposome; a vessel of a buffer; and instructions for preparing said pharmaceutical formulation.

24. The kit according to claim 23, wherein a member selected from said cyclodextrin complexed sparingly water-soluble therapeutic agent, said liposome with an ion gradient and a combination thereof are independently in dry or lyophilized form.
